(19) United States
(12) Patent Application Publication
(10) Pub. No.: US 2022/0381785 A1
(43) Pub. Date: Dec. 1, 2022

(54) Cancer Detection Method

(71) Applicant: Sienna Cancer Diagnostics Ltd, Scoresby (AU)

(72) Inventors: Matthew Hoskin, Brighton East (AU); Minesh Lalla, Moonee Ponds (AU); Shannon Turnbull, Melbourne (AU)

(21) Appl. No.: 17/666,143

(22) Filed: Feb. 7, 2022

Related U.S. Application Data
(63) Continuation of application No. 15/751,825, filed on Feb. 9, 2018, now abandoned.

(30) Foreign Application Priority Data
Aug. 19, 2015 (AU) 2015903361

(51) Int. Cl. G01N 33/574

(52) U.S. Cl. CPC G01N 33/57484; G01N 2333/901; G01N 2333/9125

(57) ABSTRACT

The present disclosure relates to methods of determining whether a subject has cancer. More particularly, the present disclosure relates to a method of determining whether a subject has cancer when a pathological assessment of cell morphology is negative for the cancer. More particularly, the present disclosure relates to a method of determining whether a morphologically normal cell is malignant. Identification of malignant cells, when a pathological assessment of cell morphology is negative for cancer, is based upon detecting the binding of an anti-telomerase antibody to clinically relevant cells.

### TECHNICAL FIELD

The present disclosure relates to methods of determining whether a subject has cancer. More particularly, the present disclosure relates to a method of determining whether a subject has cancer when a pathological assessment of cell morphology is negative for the cancer.

### BACKGROUND OF THE INVENTION

The development and progression of cancer places a significant emotional and financial burden on society.

Pathological examination (both cytology and histology) is used for diagnosis in many cancers. However, clinical diagnosis of cancer can be a difficult process, particularly in the early stages of cancer development. For example, low grade cancer pathology can have poor sensitivity leading to inconclusive or inaccurate findings.

In bladder cancer, cystoscopy and urine cytology are the most important tools in the diagnosis and follow-up of bladder cancer. However, cystoscopy with biopsy and histological assessment is generally considered the gold standard for diagnosis.

Accordingly, cytological findings often require confirmation via cystoscopy with biopsy.

A drawback of cystoscopy is that it requires an invasive procedure. In addition to being invasive and expensive, obtaining a biopsy via cystoscopy can have potential adverse outcomes for the patient. Given these limitations, it is very difficult to obtain patient samples via cystoscopy, repeatedly from a large number of individuals.

In breast cancer, mammography and biopsy with histology are commonly used as front line diagnostic tools. Massive efforts have also been invested in the identification of immunohistochemical biomarkers in breast cancer. However, the majority have not proven to be of significant value and only a few such as estrogen receptor, progesterone receptor, and Her2/neu expression are considered useful adjuncts to pathological examination (Leong and Zhuang, 2011).

A need therefore exists for a method capable of providing a more accurate, early and economically viable diagnosis of cancer. Such a method could provide assistance to clinicians in reaching an early stage diagnosis at reduced cost.

Early diagnosis of cancer, prior to invasion and metastasis, is generally associated with improved prognosis. Accordingly, there is a social and economic imperative to provide a method that can more reliably detect cancer at an early stage, so anti-cancer therapy can be administered at a time when the disease burden is mild.

### SUMMARY OF THE INVENTION

The present inventors have found that they are able to determine whether a subject has cancer when a pathological assessment of cell morphology is negative for cancer. In particular, the present inventors have found that they are able to detect cancer in cells that appear morphologically normal via pathological assessment. Accordingly, it is envisaged that the methods of the present disclosure can be used as a reflexive test to pathological assessment procedures that are negative for cancer. Thus, in a first aspect, the present disclosure relates to a method of identifying malignant cells in a sample obtained from a subject when a pathological assessment of cell morphology performed on the sample is negative for cancer, the method comprising, contacting cells from the sample with an anti-telomerase antibody and performing a pathological assessment of the cells to detect binding of the antibody to clinically relevant cells, wherein binding of the antibody to clinically relevant cells indicates the presence of malignant cells.

In an embodiment of the above aspect, the absence of antibody binding to clinically relevant cells indicates that malignant cells are not present in the sample.

In a further aspect, the present disclosure relates to a method of determining whether a subject has cancer when a pathological assessment of cell morphology performed on a sample obtained from the subject is negative for cancer, the method comprising:

i) contacting a sample obtained from the subject with an anti-telomerase antibody;

ii) performing a pathological assessment of the sample to detect binding of the antibody to clinically relevant cells in the sample;

wherein binding of the antibody to one or more clinically relevant cells in the sample indicates that the subject has cancer.

When cancer is determined using a method of the disclosure the determination may or may not be conclusive with respect to the definitive diagnosis upon which a treating physician will determine a course of treatment. The definitive diagnosis of the cancer status of a subject determined to have cancer can be validated or confirmed if warranted, such as through imaging techniques including, PET, MRI, ultrasound, CT, PET/CT. In an embodiment of the above aspects, when the cancer being determined is bladder cancer, further investigation via cystoscopy with biopsy or upper tract imaging may be used to obtain a definitive diagnosis of the cancer status.

The present disclosure can also be used as a frontline, adjunctive test, to more accurately determine the presence of malignant cells in a single procedure.

Accordingly, in a further aspect, the present disclosure relates to a method of determining whether a subject has cancer, the method comprising:

i) performing a pathological assessment of cell morphology on a sample obtained from the subject to determine the morphology of one or more clinically relevant cells in the sample;

ii) contacting a sample from the subject with an anti-telomerase antibody and performing a pathological assessment of the sample to detect binding of the antibody to clinically relevant cells in the sample;

wherein when the assessment of cell morphology is negative for cancer, binding of the antibody to one or more clinically relevant cells indicates that the subject has cancer.

In an embodiment of the above aspects, binding of the antibody to at least about 5% of clinically relevant cells in the sample indicates that the subject has cancer. Further, the pathological assessment to determine cell morphology and the pathological assessment to detect binding of the antibody to clinically relevant cells may be performed simultaneously on the same cells.

In an embodiment, the absence of antibody binding to clinically relevant cells indicates that the subject does not have cancer.

In an embodiment, the methods of the present disclosure may be used to detect cancer when a pathological assessment of cell morphology is normal, pre-malignant, metaplastic or dysplastic. In various examples, the methods of the present disclosure may be used to detect cancer when a pathological assessment of cell morphology is normal. In various examples, the methods of the present disclosure may be used to detect cancer when a pathological assessment of cell morphology is pre-malignant, metaplastic or dysplastic. For example, the methods of the present disclosure may be used to detect cancer in morphologically normal cells. For example, the methods of the present disclosure may be used to detect cancer in pre-malignant, metaplastic and/or dysplastic cells.

As will be appreciated by one of skill in the art, pathological assessment involves the assessment of individual cells in a tissue sample in conjunction with their surrounding environment. Accordingly, in performing the present method the cells are pathologically assessed to detect binding of an anti-telomerase antibody to clinically relevant cells. Pathological assessment of telomerase allows telomerase stained cell types which are known to be non-cancerous, or unrelated to the cancer being investigated, to be excluded from the assessment based on their morphology. For example, the methods of the present disclosure comprise excluding non-clinically relevant cells from the pathological assessment.

Excluded cells are considered not clinically relevant to determining whether a subject has cancer. The excluded cells will depend on the cancer being detected. More specifically, the skilled person will be aware of cell types in a sample related to a particular cancer. Examples of excluded cells include, but are not necessarily limited to, one or more or all of T-cells, B-cells, neutrophils, macrophages, granulocytes, dendritic cells, mast cells, memory-cells, plasma cells, eosinophils, seminal vesicle cells, and sperm and squamous cells. For example, the cells listed above will be excluded when assessing bladder cancer using the methods of the disclosure.

In another example, the methods of the present disclosure further comprise prescribing treatment of a subject for cancer when binding of the antibody to clinically relevant cells is detected.

In another example, the anti-telomerase antibody is monoclonal, polyclonal, bispecific, chimeric, recombinant, anti-idiotypic, humanized, single-chain antibody molecule, or antigen-binding fragments thereof.

Examples of antibodies suitable for use in the disclosure include, but are not limited to, SCD-A7, 2D8, C-12, H-231, anti-telomerase catalytic subunit, 10E9-2, 2C4, and tel 3 36-10. In an embodiment, the antibody is SCD-A7 or a telomerase binding fragment thereof.

In an embodiment, the pathological assessment is a cytological assessment. In this embodiment, the sample may be a fluid sample. For example, the fluid sample may be selected from the group consisting of urine, bladder washings, bladder scrubbings, blood, sputum, cerebrospinal fluid, pleural effusions, fine needle aspirate, cell suspension. In these examples, the cancer may be selected from the group consisting of bladder cancer, thyroid cancer, breast cancer, cervical cancer. For example, the methods of the present disclosure may be used to detect bladder cancer.

In an embodiment, the pathological assessment is a histological assessment. In this embodiment, the sample may be a tissue sample. For example, the tissue sample may be selected from the group consisting of bladder, pancreas, liver, gall bladder, thyroid, ovary, lymph node, breast, cervix, lung, biliary tree, pancreas, lung, kidney, prostate, colon, stomach, oesophagus and brain. In these examples, the cancer may be selected from the group consisting of bladder cancer, pancreatic cancer, liver cancer, gall bladder cancer, thyroid cancer, breast cancer, lung cancer, mesothelioma, cervical cancer, ovarian cancer, kidney cancer, prostate cancer, colorectal cancer, stomach cancer, oesophageal cancer, brain cancer.

In an embodiment the cancer can be any cancer where clinically relevant cells may be present which can result in a negative pathological assessment for the cancer. For example, the methods of the present disclosure may be used to detect bladder cancer.

In an embodiment, the methods of the present disclosure may be used to detect carcinoma in-situ. For example, the methods of the present disclosure may be used to detect bladder carcinoma in-situ. For example, the methods of the present disclosure may be used to detect ductal carcinoma in-situ.

The present disclosure is not to be limited in scope by the specific embodiments described herein, which are intended for the purpose of exemplification only. Functionally-equivalent products, compositions and methods are clearly within the scope of the disclosure, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The disclosure is hereinafter described by way of the following non-limiting Examples and with reference to the accompanying figures.

### BRIEF DESCRIPTION OF THE ACCOMPANYING DRAWINGS

FIG. 1: No cellular staining was observed in sample WH11-107 (clinically negative; FIG. 1A). First evidence of immunostaining of telomerase and its clinical correlation shown in clinically positive sample WH11-122 using the anti-hTERT (Clone 2C4) antibody (FIG. 1B). Positive nuclear staining was observed in 40-75% of the urothelial cells present, under optimal antibody concentrations.

FIG. 2: Positive and negative cell types observed in high grade (Panel A and B) and low grade (Panel C and D) clinical samples. Atypical urothelial cells stained nuclear positive by telomerase immunostaining (Panel A and B). Cytologically normal looking urothelial cells stained nuclear positive by telomerase immunostaining (Panel C) and within the same sample unstained cytologically normal looking urothelial cells (Panel D, inset arrow).

FIG. 3: Cells stained for telomerase hTERT protein from a clinical sample of a patient with low grade (G1) bladder cancer. Panel A: Squamous cell (not from bladder); Panel B: Normal bladder cells (and small brown blood cell); Panel C: Normal-looking bladder cell positive for Sienna test; Panel D: Cytologically abnormal bladder cell positive for telomerase immunostaining.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cancer diagnostics, pathology, cytology, histology, immunohistochemistry, protein chemistry, antibodies, biochemistry and molecular biology).

Unless otherwise indicated, the immunoassay, sample preparation, and immunological techniques referred to in the present disclosure are standard procedures, well known to those skilled in the art. Such techniques are described and explained throughout the literature in sources such as, Perbal, (1984), Sambrook et al., (1989), Brown, (1991), Glover and Hames (1995 and 1996), Ausubel et al., (1988), Harlow and Lane (1988), Coligan et al., (1994).

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−10%, more preferably +/−5%, more preferably +/−1%, more preferably +/−0.5% of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Pathological Assessment

“Pathological assessment” is used in the context of the present disclosure to refer to an assessment that seeks to identify malignant cells in a sample by visually assessing the cells in the sample. As will be apparent to one of skill in the art, pathological assessment is not a test per se but a pathology consultation based on a particular sample or sample set. Pathological assessment procedures are complex and require expertise and care in sample collection to provide a correct assessment.

In the context of bladder cancer, pathological assessment can be used alongside, or as a reflex to, cystoscopy for the detection of recurrence or the diagnosis of bladder cancer.

In the context of breast cancer, pathological assessment can be used alongside, or as a reflex to, mammography, ultrasound and other imaging techniques for the detection of recurrence or the diagnosis of breast cancer.

In the context of oesophageal cancer, pathological assessment can be used alongside, or as a reflex to, endoscopy for the detection of recurrence or the diagnosis of oesophageal cancer.

The present disclosure refers to a “pathological assessment of cell morphology” and a “pathological assessment to detect binding of an anti-telomerase antibody to clinically relevant cells”. In an example, these assessments are performed separately. In another example, these assessments are performed sequentially or simultaneously. In another example, these assessments are performed sequentially or simultaneously on the same cells.

A “pathological assessment of cell morphology” seeks to identify malignant cells in a sample by visually assessing the cell morphology of clinically relevant cells. In the context of the present disclosure, pathological assessment of cell morphology refers to procedures that are part of the standard of care and used alongside, or as a reflex to, further investigation for the detection of recurrence or the diagnosis of cancer.

A “pathological assessment to detect binding of an anti-telomerase antibody to clinically relevant cells” seeks to identify malignant cells in a sample by assessing the binding of an anti-telomerase antibody to clinically relevant cells.

Examples of pathological assessment procedures include “cytological assessment” and “histological assessment”. These procedures are discussed further below.

### Cytological Assessment

“Cytological assessment” of cell morphology in cancer diagnostics seeks to identify malignant cells based on morphologic characteristics of individual cells. In performing a cytological assessment of cell morphology, a cell sample is typically fixed to a slide and viewed under a microscope to visually assess the morphology and cellular features. For example, breast or thyroid epithelial cells harvested from a fine needle aspirate, bladder urothelial cells harvested from a urine sample or cervical epithelial cells harvested from a Papanicolaou test (cervical smear) can be fixed to a glass slide and visually assessed via cytology. Various other examples of cell samples suitable for cytological assessment are discussed below.

Before visually assessing the slide the sample may be stained to assist in visualising morphological changes to cells and cellular components (e.g. nuclei). Exemplary stains include a haematoxylin and eosin stain or Papanicolaou stain (Pap stain).

### Histological Assessment

“Histological assessment” of cell morphology in cancer diagnostics seeks to identify malignant cells in a tissue sample based on the morphologic characteristics of cells and tissue architecture.

The term “histological assessment” is used in the context of the present disclosure to refer to the assessment of tissue, in particular the cells comprising tissue and their surrounding environment.

In performing a histological assessment of cell morphology, a tissue sample is typically fixed, sectioned and mounted on a slide before being viewed under a microscope to visually assess tissue morphology, architecture and cellular features. For example, a bladder biopsy, a breast core biopsy or an oesophageal biopsy can be obtained from a subject, fixed, sectioned and mounted on a glass slide before being visually assessed under a microscope. Various other examples of tissues suitable for histological assessment are discussed below.

Before, visually assessing a tissue section on a slide, the tissue may be stained to assist in visualising morphological changes to cells, cellular components (e.g. nuclei) and characterising resident cell types. Exemplary stains include a Haematoxylin and Eosin, Papanicolaou (Pap) and Alcian Blue Periodic Acid-Schiff stains.

Pathological Assessment of Cell Morphology that is Negative for Cancer

The methods of the present disclosure relate to determining whether a subject has cancer when a pathological assessment of cell morphology performed on a cell sample obtained from the subject is negative for cancer. The term “negative for cancer” is used in the context of the present disclosure to refer to a definitive pathological call that a sample is negative for cancer. It is envisaged that various pathological calls are considered “negative for cancer”. In an example, cells with a “normal” morphology are considered negative for cancer. In an example, the methods of the present disclosure relate to the determining whether a subject has cancer when a pathological assessment of cell morphology performed on a cell sample obtained from the subject is reported as morphologically normal. Put another way, in an example, the methods of the present disclosure relate to detecting whether morphologically normal cells are cancerous.

In another example, cells with a “pre-malignant” morphology are considered negative for cancer. Pre-malignant morphology refers to a sample that bears certain hallmarks of a cancerous phenotype but remains negative for cancer as it has yet to (and in some instances may never) progress to a malignant phenotype. For example, cells with metaplastic and dysplastic morphology may be considered pre-malignant. Exemplary pathologies with pre-malignant morphology include oral epithelial lesions, Barrett's oesophagus, gastric intestinal metaplasia and nodular hyperplasia of the prostate. In an example, the methods of the present disclosure relate to the determining whether a subject has cancer when a pathological assessment of cell morphology performed on a cell sample obtained from the subject is reported as pre-malignant pathology.

In another example, cells with a “metaplastic” morphology are considered negative for cancer. Metaplastic morphology or metaplasia is characterised by replacement of one differentiated cell type with another differentiated cell type. For example, intestinal metaplasia can occur in the oesophageal or gastric epithelium when a region of the normal epithelial lining is transformed into a metaplastic epithelium characterised by the presence of “intestinal” goblet cells. In an example, the methods of the present disclosure relate to the determining whether a subject has cancer when a pathological assessment of cell morphology performed on a cell sample obtained from the subject is reported as metaplasia. For example, the pathological assessment of cell morphology may be reported as intestinal metaplasia. In the context of an oesophageal or gastric biopsy, the pathological assessment of cell morphology may be reported as columnar epithelium with intestinal metaplasia.

In another example, cells with a “dysplastic” morphology are considered negative for cancer. Exemplary characteristics of dysplasia include anisocytosis, poikilocytosis, hyperchromatism and unusual numbers of cells in mitosis. Dysplasia is generally considered the earliest form of pre-malignant lesion which pathologists can recognize via pathologic assessment. Dysplasia can be characterised as low grade dysplasia or high grade dysplasia. The risk of low grade dysplasia transforming into high grade dysplasia, and eventually cancer is low, while high grade dysplasia represents a more advanced progression towards malignant transformation. In an example, the methods of the present disclosure relate to the determining whether a subject has cancer when a pathological assessment of cell morphology performed on a cell sample obtained from the subject is reported as dysplasia. In another example, the methods of the present disclosure relate to the determining whether a subject has cancer when a pathological assessment of cell morphology performed on a cell sample obtained from the subject is reported as low grade dysplasia. In another example, the methods of the present disclosure relate to the determining whether a subject has cancer when a pathological assessment of cell morphology performed on a cell sample obtained from the subject is reported as high grade dysplasia.

For the avoidance of doubt the present disclosure does not encompass determining whether a subject has cancer when a pathological assessment of cell morphology performed on a cell sample obtained from the subject is inconclusive for cancer. Such a pathological assessment cannot provide a definitive call that a sample is negative for cancer. For example, the methods of the present disclosure do not encompass determining whether a subject has cancer when a pathological assessment of cell morphology performed on a cell sample obtained from the subject is reported as “atypical” or “indeterminate” for cancer.

### Determining Whether a Subject has Cancer

It has now been found that a pathological assessment that is negative for cancer can be resolved to determine clinical status using a telomerase immunostaining test and further pathological assessment to detect binding of an anti-telomerase antibody to clinically relevant cells.

In an example, cells isolated from a fluid sample can be fixed to a slide and cytologically assessed to detect binding of an anti-telomerase antibody to clinically relevant cells. For example, binding of an anti-telomerase antibody can be detected in individual bladder urothelial cells obtained from a urine sample via cytological assessment. For example, binding of an anti-telomerase antibody can be detected in individual follicular cells obtained from a fine needle aspirate sample via cytological assessment. An advantage of these exemplary approaches is that binding of an anti-telomerase antibody can be detected in individual clinically relevant cells.

In another example, a tissue sample can be fixed, sectioned and mounted on a slide and histologically assessed to detect binding of an anti-telomerase antibody to clinically relevant cells. For example, binding of an anti-telomerase antibody can be detected in bladder urothelial cells in a bladder wall tissue sample obtained via biopsy via histological assessment. For example, binding of an anti-telomerase antibody can be detected in breast epithelial cells in a breast tissue sample obtained via core biopsy via histological assessment. An advantage of these exemplary approaches is that binding of an anti-telomerase antibody can be detected in individual clinically relevant cells or in cell populations while also examining the surrounding tissue architecture.

### Detecting Anti-Telomerase Antibody Binding

The present inventors have found that the binding of an anti-telomerase antibody to one or more clinically relevant cells in a sample from a subject indicates that the subject has cancer. As would be appreciated by one of skill in the art, an indication of cancer also indicates the presence of malignant cells in the sample.

Telomerase is a naturally occurring enzyme that maintains the length of telomeres at the end of a chromosome. In humans, telomerase is over-expressed in human stem cells, in germ line cells and in malignant cells. The function of telomerase is to synthesise new single stranded TTAGGG repeats at the end of each chromosome. In normal cells, telomerase plays a protective role by permitting a cell to multiply, thus preventing shortening of telomeres and avoiding cellular senescence (Bodnar et al., 1998). In contrast, albeit with the same mode of action, telomerase can also exhibit cancer-promoting properties in cells which are or may become malignant. In the absence of senescence, cells (tumours) replicate indefinitely, thereby introducing and propagating mutations (Blackburn et al., 2005). This cancer-promoting property of telomerase aids the immortality of the cell and development of cancer. Its association to cancer is evident in that its presence is observed as a common feature in nearly all tumour cells.

To detect telomerase, a cell sample from a subject is contacted with an antibody that binds telomerase, also referred to as an anti-telomerase antibody. It is considered that terms such as “contacting”, “exposing” or “applying” are terms that can, in context, be used interchangeably in the present disclosure. The term contacting, requires that the anti-telomerase antibody be brought into contact with a sample to detect whether telomerase is present in one or more cells in the sample. The binding of an antibody to telomerase indicates that telomerase is present in the cell. Further, the presence of telomerase in a sample may also be referred to as telomerase positive or positive for telomerase. The binding of an antibody to telomerase is detected via pathological assessment. For example, a light microscope may be used to detect binding of an antibody to telomerase in a clinically relevant cell.

Detecting the binding of an anti-telomerase antibody to clinically relevant cells in methods of the disclosure may be accomplished by any antibody/antigen binding detection technique known in the art where binding of the antibody to the antigen in clinically relevant cells can be detected via pathological assessment. For example, an immunoassay incorporating an anti-telomerase antibody may be used. In this example, pathological assessment is used to detect binding of the anti-telomerase antibody to telomerase. In methods of the disclosure, telomerase is the “antigen”. It is also envisaged that telomerase detection methods may be incorporated into an automated telomerase detection system. Such an automated immunoassay system would provide for the automatic processing of a sample and pathological detection of telomerase. It is envisaged that such a system would allow high throughput analysis of telomerase in samples. Exemplary automated staining platforms include Ventana Benchmark Ultra or XT platforms (Ultraview or Optiview detection system), Leica Bond III (Bond Polymer Refine detection system), Dako Autostainer (Dako Envision detection system).

### Immunoassay

For routine clinical assessment it is envisaged that methods based on an immunoassay format will be used to detect the presence of telomerase in a sample. In the context of the present disclosure, an immunoassay is a biochemical test that measures the presence or concentration of an antigen in a solution through the use of an antibody or immunoglobulin.

The antibody used in the present disclosure can be any antibody that can detect whether telomerase is present in one or more cells in the sample. Various commercially available antibodies that can detect whether telomerase is present in a cell are available for use in the methods of the disclosure. Such antibodies can be obtained from Sapphire Biosciences, Life Span Biosciences, Novus Biologicals, Australian Biosearch, Epitomics, Santa Cruz, EMD Millipore, GenWay Biotech Inc, Jomar Biosciences, Sigma-Aldrich, BioCore Pty Ltd, US Biologicals, Thermo Scientific, Life Research, Resolving Images, and Sienna Cancer Diagnostics Ltd. In an example, the antibody binds the telomerase complex (including each of human telomerase reverse transcriptase, telomerase RNA (TR or TERC), and dyskerin (DKC1) and/or Telomerase reverse transcriptase (hTERT). Preferably, the antibody is an anti-hTERT antibody. Most preferably, the antibody that binds telomerase is SCD-A7, 2D8, C-12, H-231, anti-telomerase catalytic subunit, 10E9-2, 2C4, and tel 3 36-10.

These antibodies are known in the art. For example, the anti-hTERT (tel 3) antibody is a monoclonal antibody produced from the hybridoma clone, 36-10. To purify the antibody, the IgG fraction of ascites was purified by Protein G affinity chromatography. The anti-hTERT (Clone SCD-A7) antibody is an IgM mAb, produced from the hybridoma clone, HJ123-2C4 (Masutomi et al., 2003) grown in hollow fibre cultures. In producing this antibody, amino-terminal FLAG epitope-tagged hTERT purified from baculovirus vector-infected insect cells was used as an immunogen to stimulate the production of anti-hTERT Clone SCD-A7 mAb.

The anti-hTERT (Clone 2C4) antibody is described in (Masutomi et al., 2003). In producing this antibody, amino-terminal FLAG epitope-tagged hTERT purified from baculovirus vector-infected insect cells was used as an immunogen to stimulate the production of anti-hTERT Clone 2C4 mAb. 2C4 is an IgM mAb, produced from the hybridoma clone, HJ123-2C4 grown in hollow fibre cultures.

Antibodies used in the methods of the present disclosure are also commercially available such as 2D8 (Novus NB 100-297), C-12 (Santa Cruz 377511), H-231 (Santa Cruz 7212), anti-telomerase catalytic subunit (Rockland 600-401-252), 10E9-2 (MBL M216-3), 2C4 (Novus NB100-317), SCD-A7 (Sienna Cancer Diagnostics P/N 01-5001).

In an example, the antibodies of the present disclosure are detectably labelled. Examples of detectable labels include the conjugation of a dye, fluorophore or other reporter molecule for assays, tracking or imaging.

The antibody used in the present disclosure is not limited to monovalent antibodies and multivalent antibodies represented by IgM but also includes bivalent antibodies represented by IgG, so long as they bind telomerase.

Further, the antibody used in the present disclosure is not limited to whole antibody molecules, but includes minibodies, diabodies and modified products thereof, so long as they bind telomerase.

A minibody comprises antibody fragments lacking a portion of a whole antibody (for example, whole IgG), and is not particularly limited so long as it has telomerase-binding ability. With the exception of telomerase binding ability, there are no particular limitations on the antibody fragments of the present disclosure, so long as they are portions of a whole antibody, but they preferably contain a heavy chain variable region (VH) and/or a light chain variable region (VL). Furthermore, as long as it has telomerase antigen-binding ability, part of VH and/or VL can be deleted. The variable region may be chimerized or humanized. Specific examples of the antibody fragments include Fab, Fab′, F(ab′)2, and Fv. Specific examples of minibodies include Fab, Fab′, F(ab′)2, Fv, scFv (single chain Fv), diabody, and sc(Fv)2 (single chain (Fv)2). Multimers of these antibodies (for example, dimers, trimers, tetramers, and polymers) are also included in the minibodies that can be used to detect telomerase.

A diabody is a dimer composed of two polypeptide chains, and generally, the polypeptide chains are individually linked by a linker of, for example, five residues or so, which is short enough to prevent binding between VL and VH in the same chain. VL and VH that are encoded by the same polypeptide chain have a short linker between them, and form a dimer because they cannot form a single chain variable region fragment. Therefore, diabodies have two antigen binding sites.

Preferably, the antibody that binds telomerase is monoclonal, polyclonal, bispecific, chimeric, recombinant, anti-idiotypic, humanized, single-chain antibody molecule, or antigen-binding fragments thereof.

In a preferred embodiment according to the present disclosure the method for detection of telomerase uses a telomerase-specific primary antibody. Binding of the primary antibody to telomerase can be visualised via various known methods. For example, a labelled secondary antibody that recognises the primary antibody could be used. In this example, the label could be an enzyme such as horse radish peroxidase, a radioactive isotope, a fluorescent reporter, an electrochemiluminescent tag. Binding of the labelled secondary antibody to the primary antibody would be detected via pathological assessment.

In a particular example, a sample is contacted with a telomerase-specific primary anti-hTERT antibody. The sample is then washed to remove any unbound primary antibody and then a secondary antibody specific for the primary antibody and linked to a peroxidase enzyme is applied to the sample. The sample is then washed to remove any unbound secondary antibody and 3,3′-Diaminobenzidine (DAB) is applied to the sample. The conversion of DAB into a coloured product is visualised by routine pathological assessment with the presence of a coloured product indicating that telomerase is present in the sample.

### Cancer Types

In the claimed method, the cancer may be any cancer so long as the subject cancer cells express telomerase. In an example, the cancer is bladder cancer. In various other examples, the cancer is pancreatic cancer, liver cancer, gall bladder cancer, thyroid cancer, breast cancer, lung cancer, mesothelioma, cervical cancer, ovarian cancer, kidney cancer, colorectal cancer, prostate cancer, stomach cancer, oesophageal cancer or brain cancer.

As would be appreciated by one of skill in the art, each cancer type has various characteristics associated with cancer grade. These grades are generally dictated by the level of cancer spread or invasion into the surrounding tissues. For example, the later grades of cancer or “high grade” is generally associated with a higher potential for metastasis and a poorer prognosis. High grade cancers have generally spread from the tissue or organ of origin into the surrounding tissue or throughout the body. In contrast, “low grade” cancer can be characterized as carcinoma in-situ (CIS) meaning that cells are abnormally proliferating but are still contained within the tissue or organ of origin.

In an example, the methods of the present disclosure can be used to determine whether a subject has high grade cancer.

In an example, the methods of the present disclosure can be used to determine whether a subject has low grade cancer. For example, the methods of the present disclosure can be used to determine whether a subject has carcinoma in-situ. For example, the methods of the present disclosure can be used to determine whether a subject has ductal carcinoma in-situ.

In the context of bladder cancer, “High grade” or “higher grade” bladder cancer refers to a bladder cancer that is more likely to recur and/or progress and/or become invasive in a subject, including malignant cancers with higher potential for metastasis (bladder cancer that is considered to be more aggressive). Cancers that are not confined to the bladder (i.e. muscle-invasive bladder cancer) are considered to be more aggressive bladder cancers.

Low grades of bladder cancer can be characterized as carcinoma in-situ (CIS) meaning that cells are abnormally proliferating but are still contained within the bladder. “Low grade” or “lower grade” bladder cancer refers to bladder cancer, including malignant cancers with lower potential for recurrence, progression, invasion and/or metastasis (i.e. bladder cancer that is considered to be less aggressive). Cancers that are confined to the bladder (i.e. non-muscle invasive bladder cancer, NMIBC) are considered to be less aggressive bladder cancer. In an example, the methods of the present disclosure can be used to determine whether a subject has high grade cancer.

In an example, the methods of the present disclosure can be used to determine whether a subject has high grade bladder cancer.

In an example, the methods of the present disclosure can be used to determine whether a subject has low grade bladder cancer. For example, the methods of the present disclosure can be used to determine whether a subject has bladder carcinoma in-situ.

### Sample Preparation and Analysis

In performing the methods of the disclosure a sample from a subject is required. As used herein, the term “sample” refers to a cell or population of cells or a quantity of tissue from a subject. The “sample” includes extracts, derivatives, fractions, suspensions or sections of the sample. It is considered that terms such as “sample” and “specimen” are terms that can, in context, be used interchangeably in the present disclosure. In the present disclosure, any cells or tissue can be used as the above-mentioned sample so long as it can be collected from the subject. It is contemplated that the sample used in the present disclosure be a cell or tissue sample from a human.

The appropriate sample will also depend on the pathological assessment being performed. For example, a cytological assessment requires a cell sample. Preferably, the sample is a fluid sample. The fluid sample may include a variety of biological materials selected from but not limited to the group consisting of blood (including whole blood), blood plasma, blood serum, hemolysate, lymph, synovial fluid, spinal fluid, urine, bladder washings, bladder scrubbings, cerebrospinal fluid, semen, stool, sputum, mucus, amniotic fluid, lacrimal fluid, cyst fluid, sweat gland secretion, bile, milk, tears or saliva. In other examples the fluid sample can be fine needle aspirate or a cell suspension. In the context of bladder cancer, the fluid sample is a urine sample. It is envisaged that these samples can be obtained using techniques known in the art. For example, in the context of bladder cancer, a cell sample can be obtained from a subject's urine sample. In the context of breast or thyroid cancer, the sample may be obtained via fine needle aspiration. In the context of cervical cancer, the sample may be a cell suspension prepared from a Papanicolaou test.

In an example, the cell sample is treated after removal from the subject and prior to cytological assessment. For example, the tissue may be fixed, permeabilised and/or detergent treated.

In contrast, a histological assessment requires a tissue sample. In an example, the tissue sample is obtained from the bladder, pancreas, liver, gall bladder, thyroid, breast, lung, cervix, ovary, kidney, colon, prostate, stomach, oesophagus or brain.

The tissue sample may include material obtained from biopsy or resection. It is envisaged that tissue samples used in the methods of the present disclosure can also be obtained using techniques known in the art. For example, in the context of bladder cancer, a tissue sample can be obtained via cystoscopy. In the context of stomach or oesophageal cancer, a tissue sample can be obtained via endoscopy. In the context of colon cancer, a tissue sample can be obtained via colonoscopy. In the context of breast cancer, a tissue sample can be obtained via core needle biopsy or surgical biopsy.

In an example, the tissue sample is treated after removal from the subject and prior to pathological assessment. For example, the tissue may be paraffin embedded, fixed, permeabilised and/or detergent treated.

It is envisaged that the methods of the present disclosure may be performed on multiple samples obtained from the same patient. For example, the methods of the present disclosure may be used to assess at least two, at least three, at least four, at least five, at least six samples obtained from the same patient. The multiple samples may be obtained during a single consultation or procedure.

For example, multiple tissue samples may be obtained during a single biopsy procedure. For example, a four quadrant oesophageal biopsy procedure can provide four tissue samples for assessment using the methods of the present disclosure.

Multiple biopsies can also be obtained when assessing cancer margins after surgical removal of a cancer. In an example, the methods of the present disclosure are used following surgical removal of a cancer. In this example, the methods of the present disclosure are used to determine whether a subject has cancer when a pathological assessment of cell morphology of a tissue sample obtained from cancer margins is negative for cancer.

It is also envisaged that in performing the present method, the pathological assessment of cell morphology and the pathological assessment for detection of telomerase may be performed on separate cells or tissue sections obtained from the same sample or separate cell or tissue samples obtained from the same patient.

The present inventors have found that when a pathological assessment of cell morphology is negative for cancer, the binding of an anti-telomerase antibody to these cells indicates that the subject has cancer. Accordingly, it is preferable that the same sample, in particular the same slide be used for the pathological assessment of cell morphology and the pathological assessment for detection of telomerase.

Therefore, in one example, the pathological assessment of cell morphology and the pathological assessment for detection of telomerase is performed on the same cells or tissue section obtained from a subject's sample. In an example, the pathological assessment of cell morphology and the pathological assessment to detect binding of an anti-telomerase antibody are performed simultaneously or sequentially on the same cells.

### Clinically Relevant Cells

The phrase “clinically relevant cells” refers to those cells that the pathologist is examining to determine the cancer status of the patient.

Clinically relevant cells will depend on the cancer being investigated and in the context of the present disclosure can include, ductal and lobular cells of the breast, respiratory cells of the lung, mucosal cells of the digestive tract, duct and islet cells of the pancreas, hepatocytes, follicular cells, mesothelial cells, germ cells, granulosa cells and epithelial cells of the ovary, oesophagus or stomach, glandular and basal cells of the prostate, epithelial cells of the ureter, urothelial cells, ductal and tubular cells of the kidney, endometrial cells, nerve or glial cells of the brain.

In an example, the clinically relevant cells are morphologically normal. In another example, the clinically relevant cells are pre-malignant. In another example, the clinically relevant cells are metaplastic. In another example, the clinically relevant cells are dysplastic.

The determination of cancer can be made because of the general principle that normal cells do not express telomerase while malignant cells express telomerase. However, as would be appreciated by one of skill in the art, there are exceptions to this general principle as certain non-malignant cell types also express telomerase. These cells are not considered clinically relevant and should be excluded from the assessment. Preferably, the excluded cells are selected from the group comprising T-cells, B-cells, neutrophils, macrophages, granulocytes, dendritic cells, mast cells, memory-cells, plasma cells, eosinophils, seminal vesicle cells, sperm. These cells have a distinctly different morphology from clinically relevant cells and therefore can easily be excluded visually when performing the pathological assessment.

For example, when using the present methods to determine bladder cancer, there are several cell types which one of skill in the art would visually exclude from the histological analysis. These cells include inflammatory cells such as neutrophils, macrophages and eosinophils, as well as renal tubular cells, seminal vesicle cells, sperm, and squamous cells. These cells have a distinctly different morphology from clinically relevant normal urothelial cells (bladder wall cells) and therefore can easily be excluded visually when performing the pathological assessment.

In performing the present method, telomerase can be present in more than one clinically relevant cell and indicate that a subject has cancer. In various embodiments, binding of an anti-telomerase antibody to at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 30, at least about 40, at least about 50, at least about 60, at least about 70, at least about 80, at least about 90, at least about 100, at least about 200 clinically relevant cells pathologically assessed in a sample from a subject indicates that the subject has cancer. In contrast, the absence of anti-telomerase antibody binding to clinically relevant cells indicates that malignant cells are not present in the sample.

Further, telomerase can be present in a percentage of the total number of clinically relevant cells assessed in a sample and indicate that a subject has cancer. In various embodiments, binding of an anti-telomerase antibody to at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 30%, at least about 40%, at least about 50%, at least about 60%, at least about 70%, at least about 80%, at least about 90% of clinically relevant cells assessed in a sample from a subject indicates that the subject has cancer.

For example, binding of an anti-telomerase antibody to at least about 5% of bladder urothelial cells in a tissue section obtained from a subject's tissue sample indicates that the subject has cancer.

For the avoidance of doubt, it is envisaged that binding of an anti-telomerase antibody to one cell per 20 clinically relevant cells indicates that the subject has cancer.

### Reflexive Testing

It is envisaged that the claimed method may be performed as a reflexive test. A “reflexive test” refers to a subsequent test (e.g., a second test) that is undertaken based upon the results obtained in a previous test (e.g., a first test). When determining whether a subject has cancer, pathological assessment of a sample can lead to a desire to test for another target. In the context of the present disclosure, the desire to test for another target (i.e. detect binding of an anti-telomerase antibody to clinically relevant cells) is driven by a pathological assessment of cell morphology that is negative for cancer.

### Adjunctive Testing

It is also envisaged that the claimed method may be performed as an adjunctive test. A test that provides information that adds to or assists in the interpretation of the results of other tests, and provides information useful for correcting an earlier negative cancer assessment may be classified as an adjunctive test. In a clinical setting, a pathological assessment of cell morphology may be requested to determine whether a subject has cancer. While the pathological assessment may be negative for the cancer, to assist in determining whether the subject has cancer, a further pathological assessment is performed to detect the binding of an anti-telomerase antibody to clinically relevant cells in a sample from the subject as an adjunct to the pathological assessment of cell morphology. In this context, the binding of an anti-telomerase antibody to one or more clinically relevant cells indicates that the subject has cancer, correcting the negative pathological assessment of cell morphology.

In performing adjunctive testing it is envisaged that the pathological assessment of cell morphology can be performed at or about the same time as the pathological detection of telomerase. However, these steps may be performed separately.

### Subjects

As used herein, the “subject” can be any organism which can have cancer. In a preferred embodiment, the subject is a mammal. The mammal may be a companion animal such as a dog or cat, or a livestock animal such as a horse or cow. In an embodiment, the subject is a human. Terms such as “subject”, “patient” or “individual” are terms that can, in context, be used interchangeably in the present disclosure.

If malignant cells are identified in a subject using the methods of the present disclosure the subject can be directed or prescribed treatment for cancer. For example, cancer detected using the methods of the present disclosure may be treated with a pharmacological agent. Suitable exemplary therapies include, surgery, radiotherapy, chemotherapy, immunotherapy, biologics, antibody therapy, hormone therapy, stem cell transplant, photodynamic therapy, ablative therapy and various combinations thereof.

The appropriate course of treatment will depend on the identified cancer. For example, if bladder cancer is identified in a subject, the subject may be directed treatments such as transurethral resection of bladder tumour, chemotherapy, radiotherapy, Bacillus Calmette-Guérin (BCG) immunotherapy, antibody therapy or combinations thereof. For example, if breast cancer is identified in a subject, the subject may be directed treatments such as mastectomy, chemotherapy, radiotherapy, hormone therapy (e.g. anti-estrogen therapy), antibody therapy or combinations thereof. For example, if prostate cancer is identified in a subject, the subject may be directed treatments such as prostatectomy, chemotherapy, radiotherapy, hormone therapy (e.g. anti-testosterone therapy), antibody therapy or combinations thereof.

It is envisaged that the method of the present disclosure can be used to determine whether any subject has cancer. Preferably, the method is used to determine cancer in a subject with symptoms that are indicative of cancer. For example, in the context of bladder cancer, the present method would be applicable to a subject presenting to the clinic with symptoms indicative of bladder disease such as haematuria (blood in the urine); urinary frequency urgency; burning sensation on urination.

A sample used in the present disclosure may also be obtained from a subject requiring regular surveillance to monitor for new or recurrent cancer. For example, subjects with a pre-malignant condition such as Barrett's Oesophagus or ductal carcinoma in-situ (DCIS) or cancer survivors may require regular surveillance to monitor for new or recurrent malignancy. If a histological assessment of cell morphology is negative for cancer, a clinician can obtain a tissue sample from the subject under surveillance and apply the present method to determine whether they have cancer. If cancer is identified, an appropriate treatment regimen can be established.

The appropriate time course for surveillance post therapeutic intervention will depend on the subject's risk factors and cancer staging. In the context of bladder cancer, appropriate surveillance after treatment can begin with urinary pathological assessment of cell morphology every 3 months for one to two years. Screening intervals for pathological assessment of cell morphology are extended, depending on previous cellular and cystoscopic findings. In the context of breast cancer, a mammographical assessment 6 months following surgery with annual follow-up thereafter may be appropriate. In the context of prostate cancer, appropriate surveillance can include assessment of prostate-specific antigen (PSA), digital rectal exams (DREs), and ultrasound at regular intervals.

### Diagnostic Determination

It is envisaged that in performing the claimed method particular results for pathological assessment of telomerase and pathological assessment of cell morphology will be associated with a specific diagnostic determination for each subject. The telomerase and cell morphology results that may be obtained when performing the claimed method are summarised below in Table 1.

The following results indicate that the subject has cancer and therefore therapeutic intervention is warranted:
The binding of an anti-telomerase antibody to clinically relevant cells and positive pathological assessment of cell morphology;
The binding of an anti-telomerase antibody to clinically relevant cells and negative pathological assessment of cell morphology;
Although unlikely to occur given the matching sensitivities of both positive pathological assessment of cell morphology and the binding of an anti-telomerase antibody to clinically relevant cells; the absence of anti-telomerase antibody binding to clinically relevant cells and positive pathological assessment of cell morphology.

The absence of anti-telomerase antibody binding to clinically relevant cells and negative pathological assessment of cell morphology indicates that the cells in the sample are not malignant. For example, the absence of anti-telomerase antibody binding to clinically relevant cells and a negative pathological assessment of cell morphology indicates that the subject potentially has benign/reactive changes which are not linked to cancer.

TABLE 1
Diagnostic determination and clinical outcome associated
with pathological assessment of cell morphology and
pathological assessment of telomerase results

| | Pathological | |
|---|---|---|
| Pathological | Assessment | |
| Assessment of | of cell | Diagnostic determination and |
| Telomerase | morphology | Clinical Outcome |
| +ve | +ve | Positive telomerase and pathology |
| | | results indicates the subject has |
| | | cancer. Determine appropriate |
| | | therapeutic regimen. |
| +ve | −ve | Positive telomerase result indicates |
| | | the subject has cancer. Determine |
| | | appropriate therapeutic regimen. |
| −ve | +ve | The high positive predictive value |
| | | and specificity of pathology of cell |
| | | morphology alone suggests subject |
| | | has cancer. Determine appropriate |
| | | therapeutic regimen. |
| −ve | −ve | Subject is disease free. |

In the context of the present disclosure, positive pathological assessment of cell morphology refers to the identification of cells having morphological changes indicative of cancer. Morphological changes that may be associated with cancer include enlarged nuclei with irregular size and shape, prominent nucleoli, scarce cytoplasm which may be intense or pale in colour. In contrast, negative pathological assessment of cell morphology is defined as the absence of any morphological changes indicative of cancer.

### Definitive Diagnosis

In applying the methods of the present disclosure to determine whether a subject has cancer, it is considered that a diagnostic determination regarding the presence of a cancer can be made based on the binding of an anti-telomerase antibody to one or more clinically relevant cells from a sample obtained from a subject. However, the diagnostic determination may or may not be conclusive with respect to the definitive diagnosis upon which a treating physician will determine a course of treatment. Put another way, a diagnostic determination obtained using the techniques of the disclosure would be understood by one skilled in the art to refer to the process of attempting to determine or identify a possible cancer.

The methods of the present disclosure can be used in providing assistance in assessing the risk of cancer development and would be considered to assist in making an assessment of a pre-clinical determination regarding the presence, or nature, or a predisposition or precursor to cancer. This would be considered to refer to making a finding that a subject has a significantly enhanced probability of developing cancer.

It is envisaged that the methods of the present disclosure can also be used in combination with other methods of clinical assessment of cancer known in the art in providing an evaluation of the presence of cancer or an increased risk of cancer.

The definitive diagnosis of the cancer status of a subject determined to have cancer can be validated or confirmed if warranted, such as through imaging techniques including, PET, MRI, ultrasound, CT, PET/CT. Accordingly, the methods of the present disclosure can be used in a pre-screening manner, and if warranted, a further assessment can be conducted.

### Sensitivity and Specificity

In some embodiments, the sensitivity and/or specificity are measured against a clinical diagnosis of cancer.

In various embodiments, the sensitivity achieved by the presently claimed method for determining whether a subject has cancer is at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%.

In various embodiments, the specificity achieved by the presently claimed method for determining whether a subject has cancer is at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, or at least about 95%.

### EXAMPLES

### Example 1: Clinical Samples

An ethics-controlled and approved proof of concept study on clinical material from urological patients suspected of having, or with a history of, bladder cancer (urothelial cell carcinoma) was performed in order to demonstrate the clinical diagnostic potential of telomerase hTERT protein immunostaining. Furthermore, the study aimed to demonstrate that hTERT immunostaining can differentiate samples obtained from low grade versus high grade patients.

In this study, clinically positive patients had biopsy proven bladder cancer (including both non-invasive and muscle-invasive). All stage and grade determinations were by histology. In the case where a patient had multiple bladder cancer foci at different stages/grades, the higher stage/grade was recorded.

Clinically negative patients were all asymptomatic individuals in good health with no history of genitourinary disease or all disease-free patients presenting, for the first time or under monitoring for prior bladder cancer, for the visual inspection of the bladder wall (by flexible/rigid cystoscopy).

A patient was excluded from the study if any of the following criteria were met:
a) the patient had suspicious/uncharacterised non-bladder cancer diagnosis on visual inspection (flexible/rigid cystoscopy) with no histology performed;
b) monitoring patients that had a radical cystectomy;
c) patients with adenocarcinomas and non-urothelial bladder cancers (including small cell carcinoma, carcinosarcoma, primary lymphoma, and sarcoma; and/or
d) patients with other genitourinary tumours (kidney, prostate, upper ureter).

A total of 253 samples were originally tested. Of these, 108 samples were assessed using the protocol outlined below in Example 2 where a form of epitope retrieval was used either by freeze thaw or heat-induced. Of those, 90 samples had at least one urothelial (bladder wall) cell present. The remaining 18 samples were removed from all further analyses since no relevant bladder cell wall cells (i.e.: urothelial cells) were found. Of those 90 samples, five clinically positive samples were removed from further analysis due to the lack of biopsy-proven disease. The remaining 85 samples are represented in Table 2 (all clinically positive samples—biopsy-proven) and Table 3 (all clinically negative samples—cystoscopically clear), with cytological assessment of cell morphology and telomerase immunostain results shown for each.

For the eighty-five samples analysed, the number of clinically relevant cells ranged from 5 to 3000 in both clinically positive and negative patients.
Clinically negative samples: Range 5-900 urothelial cells.
Clinically positive samples: Range 10-3000 urothelial cells.

For all patients, telomerase immunostaining and parallel-cytological assessment of cell morphology on the same sample was performed and the results recorded. Telomerase hTERT protein immunostaining and cytological assessment of cell morphology was performed blind of each other and blind of clinical status (obtained by cystoscopy+/−biopsy). Immunostaining, cytological assessment of cell morphology and cystoscopy were all performed by trained clinicians and/or pathologists, registered to perform these assays in their respective clinico-diagnostic and/or medical fields.

Scoring of the telomerase immunostaining was determined by a cytologist who scanned an adequate number of fields of view to obtain a confident assessment of the slide. The number/percentage of urothelial cells displaying nuclear staining was recorded by the reading cytologist. A cut-off value for test positivity was set at >5% of urothelial cells showing nuclear staining.

All samples (both clinically positive and negative) that were classified as atypical after cytological assessment of cell morphology are given in Table 4.

### Example 2: Collection and Processing of Samples

Voided urine from patients was either processed immediately, or maintained at 4° C. for a period no longer than 4-6 hours prior to processing. Samples were transferred into sterile 50 mL centrifuge tubes and centrifuged at 600 g for 10 minutes at 4° C. The tubes were removed and the supernatant discarded. Cell pellets were resuspended in 15 mL 1×PBS and transferred into sterile 15 mL centrifuge tubes. Samples were re-centrifuged at 600 g for 10 minutes at 4° C. and the supernatant again discarded. Cell pellets were finally resuspended in 1 ml 1×PBS prior to cell counting.

Approximately 30,000 cells per sample were transferred to sterile 15 mL centrifuge tubes and the volume adjusted to 10 mL with Shandon Cytospin Collection Fluid (Thermo Scientific, Ref No: 6768001, Lot No: 226955). Tubes were again centrifuged at 600 g for 10 minutes at 4° C. The supernatant was discarded prior to resuspending in Shandon Cytospin Collection Fluid at a ratio of 250 μL per 30,000 cells. A Shandon Cytospin 4 (Thermo Scientific, Part No: A778300101, Serial No: CY6695 1055) was used to affix the cells to glass microscope slides by centrifuging the cells at 1000 rpm for 4 minutes with low acceleration.

Slides with affixed cells were stored in a microscope slide box at 4° C. overnight before transferring the slides into the −20° C. freezer.

All staining results reported herein were performed on Ventana Benchmark XT or Ventana Benchmark Ultra automated staining platforms. Identical results have been obtained, after optimisation, on other automated staining platforms including, but not limited to, Leica Bond and Biocare intelliPATH FLX. In addition to automated staining platforms, identical results have also been obtained, after optimisation, using a manual immunostaining process.

For the manual immunostaining process, slides were post-fixed in cold 50% acetone: methanol for 10 minutes. Slides were washed in 1× Phosphate buffered saline (PBS), pH 7.4, (Catalogue Number: 10010-023, 5×500 mL Gibco® by Life Technologies) to remove residual fixative and then placed in a staining dish containing citrate buffer pH 6.0. The slides are treated to a manual form of antigen retrieval for 30 minutes at 95° C. in a microwave oven. Slides are again washed in 1×PBS, pH 7.4. Post retrieval, slides were blocked with 5% BSA in 1×PBS with 0.5% Tween 20 for 1 hour, prior to incubating with the telomerase-specific primary anti-hTERT antibody at room temperature for 2 hours and washed in 1×PBS with 0.5% Tween 20. Slides are treated with a post-primary antibody block, Novocastra Post Primary (Ref: RE7159, Lot #6012593, 125 mL, Leica Microsystems) for 1 hour at room temperature and again washed in 1×PBS with 0.5% Tween 20. Slides are incubated with the secondary antibody Novocastra Novolink Polymer (Ref: RE7161, Lot #6012594, 125 mL, Leica Microsystems) for 30 minutes at room temperature and washed in 1×PBS with 0.5% Tween 20 prior to incubating with 3,3′-Diaminobenzidine (DAB) enhanced liquid substrate (Product Number: D3939, Sigma) for 2 minutes at room temperature. Slides are rinsed in 1×PBS, pH 7.4 to stop the reaction. Slides are then incubated in 0.5% Methyl Green solution at 60° C. for 5 minutes (Product Code: M8884-25G, Lot #MKBD8768V, 25 g, Sigma) and washed in tap water. Slides are dehydrated 0.05% (v/v) glacial acetic acid in acetone, then 95% ethanol, 100% ethanol followed by a final step in xylene. Slides are mounted with Ultramount No: 4 Mounting Media (Product Code: II065C, Batch #1305141450, 100 mL) and immediately a coverslip is applied before drying for 1 hour. Slides are observed under a light microscope.

For the automated immunostaining process, slides were post-fixed in cold 50% acetone: methanol for 10 minutes. Slides were washed/dipped in Ventana Antibody Dilution Buffer (Ventana Medical Systems, Inc Cat No: ADB250) to remove residual fixative and then placed on the automated staining platform and antigen retrieved on-board at 95° C. for 8 minutes. Post retrieval, slides were blocked with Ventana Discovery reagent (Ventana Medical Systems, mnc, Cat No: 760-108) for 4 minutes, prior to incubating with the telomerase-specific primary anti-hTERT antibody at 36° C. for 32 minutes. The remaining steps relied on standard Ventana platform settings using Ventana Discovery and Ventana UltraView Universal DAB Detection Kit (Ventana Medical Systems Inc, Cat No: 760-500). Hematoxylin counterstaining was performed off-board in a standard dip-dunk stainer. Counter staining could also be performed on-board to obtain identical results.

The results herein were obtained using the anti-hTERT (Clone 2C4) antibody described in (Masutomi et al., 2003).

TABLE 2
Cytological assessment of cell morphology and telomerase immunostain
results in clinically positive samples (biopsy proven).

Immunostaining result

| | | Cytological | | % |
|---|---|---|---|---|
| | | assessment | | Urothelial |
| | | of cell | Test | cells |
| # | ID | morphology* | result | stained |
| 1 | WH12-263\hat{ } | Benign | Positive | 80 |
| 2 | AUA12-055 | Positive | Positive | 45 |
| 3 | WH12-278 | Positive | Positive | 12.5 |
| 4 | AUA12-057 | Atypical | Positive | 10 |
| 5 | AUA12-059\hat{ } | Positive | Positive | 90 |
| 6 | WH12-287 | Positive | Positive | 75 |
| 7 | WH12-307\hat{ } | Benign | Positive | 50 |
| 8 | WH12-320 | Positive | Positive | 40 |
| 9 | AUA12-087 | Positive | Positive | 100 |
| 10 | WH12-373 | Benign | Negative | 0 |
| 11 | WH12-390 | Benign | Negative | 0 |
| 12 | AUA12-088 | Positive | Positive | 80 |
| 13 | AUA12-090 | Benign | Negative | 1 |
| 14 | WH12-401 | Benign | Negative | 1 |
| 15 | WH12-406 | Atypical | Positive | 30 |
| 16 | WH12-407 | Benign | Negative | 0 |
| 17 | WH12-411 | Benign | Negative | 0 |
| 18 | RMH12-001 | Benign | Positive | 10 |
| 19 | WH12-422 | Positive | Positive | 95 |
| 20 | RMH12-010 | Atypical | Positive | 10 |
| 21 | WH12-424 | Benign | Positive | 10 |
| 22 | AUA12-101 | Positive | Positive | 80 |

*An “atypical” cytological assessment of cell morphology indicates that cells in the sample have lost their normal appearance but have not reached the level of abnormality of malignant cells.
\hat{ }Sample re-stained

TABLE 3
Cytological assessment of cell morphology and telomerase immunostain
results in clinically negative samples (biopsy proven).

Immunostaining result

| | | Cytological | | % |
|---|---|---|---|---|
| | | assessment | | Urothelial |
| | Sienna | of cell | Test | cells |
| # | ID | morphology* | result | stained |
| 1 | AUA12-054 | Benign | Negative | 1 |
| 2 | WH12-268 | Benign | Positive | 10 |
| 3 | WH12-269 | Benign | Negative | 2 |
| 4 | WH12-274 | Benign | Negative | 0 |
| 5 | WH12-276 | Benign | Negative | 2 |
| 6 | WH12-279 | Benign | Negative | 0 |
| 7 | WH12-282 | Benign | Negative | 0 |
| 8 | AUA12-058 | Atypical | Positive | 20 |
| 9 | WH12-283 | Benign | Positive | 20 |
| 10 | WH12-284 | Benign | Negative | 1 |
| 11 | WH12-285 | Benign | Positive | 10 |
| 12 | WH12-286 | Benign | Negative | 0 |
| 13 | AUA12-060 | Benign | Negative | 1 |
| 14 | WH12-288 | Benign | Positive | 10 |
| 15 | WH12-290 | Benign | Negative | 0 |
| 16 | WH12-291 | Atypical | Positive | 15 |
| 17 | WH12-294 | Positive | Positive | 50 |
| 18 | WH12-295 | Benign | Negative | 0 |
| 19 | WH12-297 | Benign | Negative | 0 |
| 20 | WH12-298 | Benign | Negative | 0 |
| 21 | WH12-299 | Benign | Negative | 5 |
| 22 | WH12-301 | Benign | Negative | 1.5 |
| 23 | WH12-302 | Benign | Positive | 10 |
| 24 | WH12-304 | Benign | Negative | 1 |
| 25 | WH12-305 | Benign | Negative | 1 |
| 26 | WH12-309 | Benign | Negative | 2 |
| 27 | WH12-312 | Benign | Negative | 2 |
| 28 | WH12-313 | Benign | Positive | 20 |
| 29 | WH12-318 | Atypical | Positive | 10 |
| 30 | WH12-319 | Benign | Negative | 2 |
| 31 | WH12-322 | Benign | Negative | 1 |
| 32 | WH12-324 | Benign | Negative | 5 |
| 33 | WH12-326 | Benign | Negative | 0 |
| 34 | WH12-327 | Benign | Negative | 0 |
| 35 | WH12-329 | Benign | Negative | 1 |
| 36 | WH12-330 | Benign | Negative | 1 |
| 37 | WH12-331 | Benign | Negative | 2 |
| 38 | WH12-332 | Benign | Negative | 1 |
| 39 | WH12-336 | Benign | Negative | 1 |
| 40 | WH12-337 | Benign | Negative | 1 |
| 41 | WH12-339 | Benign | Negative | 2 |
| 42 | WH12-341 | Benign | Negative | 2 |
| 43 | WH12-342 | Benign | Negative | 2 |
| 44 | AUA12-061 | Benign | Negative | 5 |
| 45 | AUA12-064 | Benign | Negative | 2 |
| 46 | AUA12-065 | Benign | Negative | 5 |
| 47 | AUA12-092 | Benign | Negative | 1 |
| 48 | WH12-368 | Benign | Negative | 2 |
| 49 | WH12-377 | Benign | Negative | 1 |
| 50 | WH12-378 | Benign | Positive | 10 |
| 51 | WH12-387 | Benign | Negative | 0 |
| 52 | WH12-396 | Benign | Negative | 0 |
| 53 | AUA12-093 | Benign | Negative | 5 |
| 54 | AUA12-095 | Benign | Negative | 1 |
| 55 | WH12-400 | Benign | Negative | 0 |
| 56 | WH12-410 | Benign | Negative | 0 |
| 57 | WH12-412 | Benign | Positive | 10 |
| 58 | RMH12-006 | Benign | Negative | 2 |
| 59 | RMH12-007 | Atypical | Negative | 5 |
| 60 | RMH12-008 | Benign | Positive | 20 |
| 61 | AUA12-098 | Benign | Positive | 20 |
| 62 | RMH12-009 | Benign | Positive | 10 |
| 63 | AUA12-099 | Benign | Negative | 0 |

*An “atypical” cytological assessment of cell morphology indicates that cells in the sample have lost their normal appearance but have not reached the level of abnormality of malignant cells.

TABLE 4
Clinical status, cytological assessment of cell morphology and
telomerase immunostain results in all samples (both clinically
positive and negative) that had an atypical cytology.

| | | | | Immunostaining result | | |
|---|---|---|---|---|---|---|
| | | | Cytological | | % | |
| | | | assessment | | Urothelial | Follow up |
| | Sienna | Clinical | of cell | Test | cells | clinical |
| # | ID | status | morphology* | result | stained | diagnosis |
| 1 | WH12-174\hat{ } | Positive | Atypical | Positive | 80 | |
| 2 | WH12-178 | Negative | Atypical | Negative | 0 | |
| 3 | WH12-197 | Negative | Atypical | Negative | 0 | |
| 4 | WH12-233 | Negative | Atypical | Negative | 0 | |
| 5 | WH12-234 | Negative | Atypical | Negative | 0 | |
| 6 | AUA12-057 | Positive | Atypical | Positive | 10 | |
| 7 | AUA12-058 | Negative | Atypical | Positive | 20 | Positive |
| 8 | WH12-289 | Positive | Atypical | Positive | 75 | |
| 9 | WH12-291 | Negative | Atypical | Positive | 15 | Positive |
| 10 | WH12-318 | Negative | Atypical | Positive | 10 | Cystectomy |
| 11 | WH12-353 | Negative | Atypical | Negative | 0 | |
| 12 | WH12-361 | Negative | Atypical | Negative | 0 | |
| 13 | WH12-374 | Negative | Atypical | Negative | 1 | |
| 14 | WH12-406 | Positive | Atypical | Positive | 30 | |
| 15 | RMH12-007 | Negative | Atypical | Negative | 5 | |
| 16 | RMH12-010 | Positive | Atypical | Positive | 10 | |

*An “atypical” cytological assessment of cell morphology indicates that cells in the sample have lost their normal appearance but have not reached the level of abnormality of malignant cells.
\hat{ }Sample re-stained

### Example 3: Immunostaining of Telomerase in Bladder Cancer Samples Correlates with Disease

85 clinical samples were processed onto microscope slides and stained using the protocol described above. Minor adjustments to antibody conditions or cell sample preparation were empirically determined on a sample-to-sample basis. Every sample that received telomerase immunostaining also underwent standard cytological assessment of cell morphology on the same sample. Cytological assessment of cell morphology was scored as positive, negative, or atypical.

Cytological assessment of cell morphology and telomerase immunostaining results were compared in clinically positive (Table 2) and clinically negative samples (Table 3). 22 clinically positive results were assessed with 16 having telomerase positive staining. Of the 6 remaining clinically positive samples 6 were identified as benign following cytological assessment of cell morphology. Accordingly, telomerase was indicative of 16 out of 22 (72%) malignant bladder cancers.

63 clinically negative samples were assessed with 48 (76%) having telomerase negative staining. Of the 15 remaining clinically negative samples, 11 (73%) were identified as benign following cytological assessment of cell morphology. The remaining 4 (27%) were identified as indeterminate (n=3) or positive (n=1) following cytological assessment of cell morphology.

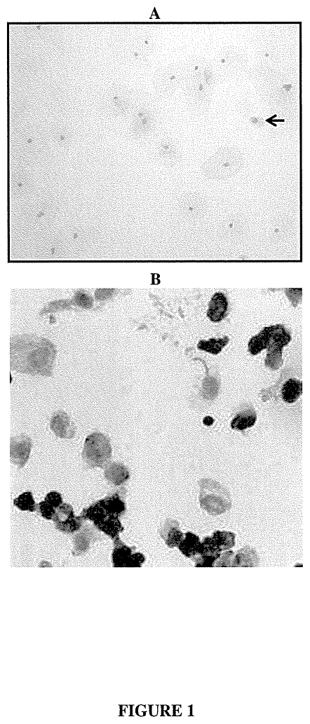

Telomerase staining correlating with disease is shown in FIG. 1. No cellular staining was observed in sample WH11-107 (clinically negative; FIG. 1A). In contrast, significant cellular staining was observed in the clinically positive sample WH11-122 (FIG. 1B). In this sample, positive staining, in the form of strong nuclear staining was observed in 40-75% of the urothelial cells present, under optimal antibody concentrations. It was interesting to note that, not all the urothelial cells present in this clinical sample stained for the presence of telomerase hTERT protein, suggesting that not all cells within the sample were cancerous.

### Example 4: Resolving False-Negative and Indeterminate Cytological Assessment of Cell Morphology

Every sample (n=85) that received telomerase immunostaining also underwent standard cytological assessment of cell morphology on the same sample. Cytological assessment of cell morphology was scored as positive, negative, or atypical. All samples (both clinically positive and negative) that were classified as atypical following cytological assessment of cell morphology (n=16) are shown in Table 4 together with the associated scoring of the telomerase immunostaining.

The sixteen samples that had atypical cytology were evaluated using one of two different immunostaining protocols, with and without a form of epitope retrieval. There were eight samples in each protocol group, each giving similar results. Of the 16 samples, 5 samples were clinically positive and 11 were clinically negative. The telomerase immunostaining method assessed 5 of these 5 samples as positive (with >5% urothelial cells staining nuclear positive).

Of the 11 clinically negative samples, 8 were shown to be negative by the immunostain test, and three were shown to be positive. Of these three, two patients in longitudinal follow-up were later assessed to be clinically positive (biopsy-proven; clinical samples AUA12-058; WH12-291). The other sample is currently pending confirmatory clinical follow-up (WH12-318). However, this patient underwent cystectomy. Performance of this procedure indicates that the patient was positive for bladder cancer.

On the basis of current clinical status, the performance of the telomerase immunostain test is 80% in specificity and 83% in sensitivity, against cystoscopy. Furthermore, the immunostain result provided a correct diagnostic indicator (relative to cystoscopy) in at least 94% of cases (15 of 16), (likely 100% of cases; 16 of 16 as WH12-318 underwent cystectomy) where cytological assessment of cell morphology gave an inconclusive reading.

### Example 5: Improving Diagnostic Readout

The unique method of preparing and immunostaining the clinical sample for the presence of telomerase, and simultaneously cytologically assessing the binding of an anti-telomerase antibody to clinically relevant cells and cytologically assessing cell morphology on a per-cell basis allowed for significant diagnostic improvements over cytological assessment of cell morphology alone.

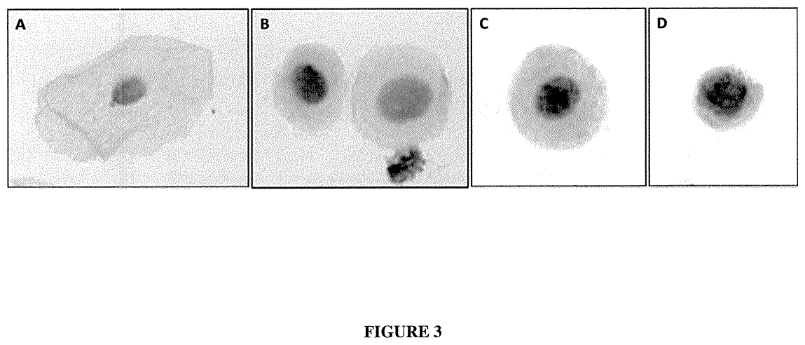

In FIG. 3, the power of resolving an indeterminate cytological assessment of cell morphology result, or salvaging a false-negative cytological assessment of cell morphology result, through telomerase immunostaining on a per-cell basis, is shown. In this figure, the cells shown in all Panels are from a low grade bladder cancer clinical sample optimally immunostained for telomerase.

In Panel A, a non-bladder squamous cell is shown. This cell is not from the bladder and is visually excluded by trained cytologists from all diagnostic determinations. It serves as a negative immunostaining control in this clinical sample. As shown in panel A, the squamous cell is completely devoid of nuclear staining, as expected.

In Panel B, normal urothelial cells are shown. Both cells shown have well defined shapes and nuclear: cytoplasmic ratios. To a trained cytologist, these cells look completely normal, and would be appropriately defined as cytology negative following cytological assessment of cell morphology. The absence of nuclear immunostaining for telomerase hTERT protein suggests that these urothelial cells, although they were found in the voided urine of a patient known to have low grade bladder cancer, are very likely to be normal urothelial cells from a normal area of the bladder wall.

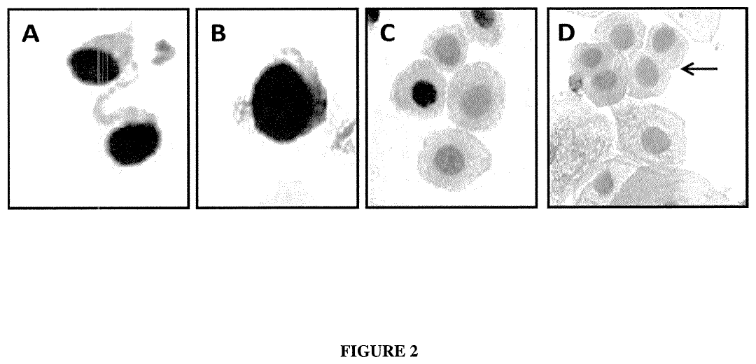

Panel C shows a morphologically normal urothelial cell of defined shape and nuclear: cytoplasmic ratio, yet in this case, strong nuclear immunostaining for telomerase hTERT protein is shown. This cytologically negative urothelial cell is, on the contrary, expressing abnormal levels of nuclear telomerase and is thus extremely likely to be an early stage malignant cell that has yet to show any morphological abnormalities. This conclusion is supported by the strong clinical correlation between telomerase hTERT immunostaining and clinical outcome shown in FIG. 2.

In the absence of telomerase immunostaining on the very same sample and the very same cell on which a determination was made based on a cytological assessment of cell morphology, this cell would have been defined as normal or non-cancer by a trained cytologist and/or pathologist. This would be incorrect and result in a false-negative call on that specific cell. Accordingly, the per-cell immunostaining of telomerase correctly determined this call.

In Panel D, a urothelial cell showing minor atypical traits, not strong enough to be called cytologically positive following cytological assessment of cell morphology by a trained cytologist, displays strong nuclear telomerase immunostaining. This is an example where individual cells classified as indeterminate after cytological assessment of cell morphology can still be resolved successfully by telomerase hTERT immunostaining under optimal conditions.

### Example 6: Identifying Malignant Cells with Normal Morphology

In a first experiment, three clinically negative samples, classified as inconclusive following cytological assessment of cell morphology were shown to be positive by the immunostain test. Of these three samples, two patients in longitudinal follow-up were later assessed to be clinically positive (biopsy-proven; clinical samples AUA12-058; WH12-291). The other sample is currently pending confirmatory clinical follow-up (WH12-318), however, this patient underwent cystectomy. Performance of this procedure indicates that the patient was positive for bladder cancer.

In further experiments, ethically-approved clinical specimens were obtained through public and private hospital collection sites and biobanks. Voided urine or tissue samples (collected in sterile saline) from patients were either processed immediately, or maintained at 4° C. for a period no longer than 48 hours prior to processing. If the tissue sample was whole and not collected as a fine needle aspirate, it was minced into a cell suspension. Samples were transferred into sterile 50 mL centrifuge tubes and centrifuged at 470 g for 10 minutes at 4° C. The tubes were removed and the supernatant discarded. Cell pellets were resuspended in 10 mL 1×PBS and re-centrifuged at 470 g for 10 minutes at 4° C. and the supernatant again discarded. Cell pellets were finally resuspended in 1 ml 1×PBS prior to cell counting.

The 1 ml cell suspension was adjusted to 10 mL with Shandon Cytospin Collection Fluid (Thermo Scientific, Ref No: 6768001, Lot No: 226955). Tubes were again centrifuged at 470 g for 10 minutes at 4° C. The supernatant was discarded prior to resuspending in Shandon Cytospin Collection Fluid at a ratio of 250 μL per 30,000 cells. A Shandon Cytospin 4 (Thermo Scientific, Part No: A778300101, Serial No: CY6695 1055) was used to affix the cells to glass microscope slides by centrifuging the cells at 1000 rpm for 4 minutes with low acceleration. Slides with affixed cells were stored at 4° C. until use.

For the automated immunostaining process, slides were placed on the automated staining platform and antigen retrieved on-board at 95° C. for 8 minutes. Post retrieval, the Pre-primary Peroxide Inhibitor was applied, prior to incubating with the telomerase-specific primary anti-hTERT antibody at 37° C. for 36 minutes. The Ventana Discovery reagent (Ventana Medical Systems, Inc, Cat No: 760-108) was required for a post-blocking step of 16 minutes. The remaining steps relied on standard Ventana platform settings using Ventana OptiView DAB IHC Detection Kit (Ventana Medical Systems Inc, Cat No: 760-700). Hematoxylin counterstaining was performed using Hematoxylin II (Ventana Medical Systems Inc, Cat No: 790-2208).

The samples were analysed by two methods—cytological and immunocytochemical assessment. The results were then matched with the clinical status of the patient (cystoscopy and/or biopsy proven). The Papanicolaou stain (Pap stain) was used to determine the morphological characteristics of individual cells, while the immunoassay used the anti-hTERT antibody to detect the presence of telomerase (indicated by brown staining in the nucleus of the clinically relevant cells). Where a sample is defined as benign, it was assessed that only morphologically normal cells were identified. For samples that have been classified as malignant based on cytological assessment, they may contain both malignant and morphologically normal cells.

Analysis revealed nuclear telomerase immunostaining in morphologically normal cells from 18 samples clinically positive for bladder cancer (Table 5). Two of these samples were previously classified via cytology as benign or atypical (Table 5). 16 of these samples were previously classified via cytology as malignant or atypical (Table 5).

In the data provided, we have identified samples that would be given a negative cancer diagnosis based on morphology alone. In the cases where the cytology assessment was benign, the patient would have been given a negative diagnosis. Sample RMH16-320 was assessed as benign, however there was telomerase detected by the immunoassay. The detection of telomerase in the morphologically normal cells has correctly matched up with the clinical result (confirmed by cystoscopy and/or biopsy). The positive result with the anti-hTERT antibody has improved the sensitivity of detecting cancer.

TABLE 5
Clinical status, cytological assessment of cell
morphology and telomerase immunostain results in
samples clinically positive for bladder cancer.

| | | | | Immunostaining result | |
|---|---|---|---|---|---|
| | | | Cytological | | Staining in |
| | | | assessment | | morphologically |
| | Sienna | Clinical | of cell | Test | normal urothelial |
| # | ID | status | morphology* | result | cells |
| 1 | RMH15-074 | Positive | Atypical | Positive | No |
| 2 | RMH15-106 | Positive | Malignant | Positive | No |
| 3 | RMH15-116 | Positive | Malignant | Positive | Yes |
| 4 | RMH15-158 | Positive | Malignant | Positive | No |
| 5 | RMH15-169 | Positive | Atypical | Positive | No |
| 6 | RMH15-183 | Positive | Malignant | Positive | No |
| 7 | RMH15-189 | Positive | Malignant | Positive | No |
| 8 | RMH15-222 | Positive | Malignant | Positive | No |
| 9 | RMH15-249 | Positive | Atypical | Positive | No |
| 10 | RMH16-096 | Positive | Malignant | Positive | Yes |
| 11 | RMH16-285 | Positive | Atypical | Positive | Yes |
| 12 | RMH16-309 | Positive | Atypical | Positive | Yes |
| 13 | RMH16-320 | Positive | Benign | Positive | Yes |
| 14 | RMH16-341 | Positive | Benign | Positive | Yes |
| 15 | AUA14-147 | Positive | Malignant | Positive | Yes |
| 16 | AUA14-150 | Positive | Malignant | Positive | Yes |
| 17 | AUA15-154 | Positive | Malignant | Positive | Yes |
| 18 | AUA15-213 | Positive | Malignant | Positive | Yes |

Analysis revealed nuclear telomerase immunostaining in morphologically normal cells from two samples clinically positive for thyroid cancer (Table 6). Both of these samples were previously classified via cytological assessment of cell morphology as benign (Table 6).

TABLE 6
Clinical status, cytological assessment of cell
morphology and telomerase immunostain results in
samples clinically positive for thyroid cancer.

| | | | | Immunostaining result | |
|---|---|---|---|---|---|
| | | | Cytological | | Staining in |
| | | | assessment | | morphologically |
| | Sienna | Clinical | of cell | Test | normal urothelial |
| # | ID | status | morphology* | result | cells |
| 1 | 15MH0001 | Positive | Benign | Positive | Yes |
| 2 | 15MH0112 | Positive | Benign | Positive | Yes |

Analysis revealed nuclear telomerase immunostaining in morphologically normal cells from two additional samples clinically positive for pancreatic cancer (Table 7). Both of these samples were previously classified via cytological assessment of cell morphology as benign or a typical (Table 7).

TABLE 7
Clinical status, cytological assessment of cell morphology
and telomerase immunostain results in samples clinically
positive for pancreatic cancer.

| | | | | Immunostaining result | |
|---|---|---|---|---|---|
| | | | Cytological | | Staining in |
| | | | assessment | | morphologically |
| | Sienna | Clinical | of cell | Test | normal urothelial |
| # | ID | status | morphology* | result | cells |
| 1 | 15MH0226 | Positive | Benign | Positive | Yes |
| 2 | 15MH0265 | Positive | Atypical | Positive | Yes |

These data suggest that telomerase may be indicative of malignant cells that have a normal morphology. These data show that telomerase may be indicative of malignant cells that have a normal morphology but were classified as malignant or atypical by cytology. This provides greater confidence in achieving the correct clinical result. Accordingly, patients with pathology that is negative for cancer which have provided samples that have positive telomerase staining may be subject to further testing, for example, in the case of bladder cancer, cystoscopy with biopsy and histological assessment. Patients clinically negative for cancer, which have provided samples that have positive telomerase staining, may be placed under increased clinical surveillance.

### Example 7: Telomerase Staining in Clinical Setting to Diagnose Bladder Cancer

A patient presents to a clinic with symptoms indicative of bladder disease such as haematuria (blood in the urine), urinary frequency urgency or burning sensation on urination. While, these symptoms can be caused by other, much less serious conditions than cancer, such as a urine infection, they are characteristic of bladder cancer.

Accordingly, a urine sample is obtained from the patient and sent for cytological assessment of cell morphology and telomerase immunostaining. If the results of the cytological assessment of cell morphology are negative for bladder cancer, the clinician can use the telomerase immunostaining results to determine whether the patient has bladder cancer.

If the sample is telomerase positive, the improved sensitivity of the telomerase assay over cytology at least warrants cystoscopic investigation for bladder cancer.

A cystoscopy can then be performed on the patient and if bladder cancer is subsequently identified, the appropriate treatment regimen can be established.

### Example 8: Telomerase Staining and Bladder Cancer Surveillance

Patients presenting with similar symptoms to those discussed above that remain unresolved or patients at increased risk of cancer development or relapse may require regular surveillance for the development of cancer.

Periodic tissue samples can be obtained from these patients and sent for histological assessment of cell morphology and telomerase immunostaining. If the results of the histological assessment of cell morphology are negative for cancer and the sample is telomerase positive, the clinician can a request a further cystoscopy and/or establish an appropriate treatment regimen. If the results of the histological assessments are negative, the clinician can continue to monitor the subject over time and request a further follow up cystoscopy with histology and telomerase staining at a later date.

### Example 9: Telomerase Staining in Clinical Setting to Diagnose Breast Cancer

A patient presents to a clinic for routine mammographic screening for breast cancer. Minor calcification is observed that is of low suspicion radiographically.

Ductal carcinoma in-situ is suspected and a fine needle aspiration sample is obtained from the patients breast. The fine needle aspiration sample can be sent for cytological assessment of cell morphology and telomerase immunostaining.

If the results of the cytological assessment of cell morphology is negative for breast cancer, the clinician can use the telomerase immunostaining results to determine whether the patient has breast cancer.

If the sample is telomerase positive, a core biopsy sample can be obtained to provide additional confirmation that the subject has cancer and/or an appropriate treatment regimen can be established.

### Example 10: Comparative Immunostaining of Telomerase in Bladder Cancer Samples

Telomerase staining was compared in clinical samples using the methods outlined above in Example 2. Comparative immunostaining was performed using SCD-A7, Novus 2C4, Novus NB 100-297, Santa Cruz 377511, Santa Cruz 7212, Rockland 600-401-252 and MBL M216-3 antibodies. Comparative immunostaining results are shown in Table 8.

### Example 12: Slide Reading Algorithm

Two slide reading algorithms have been evaluated utilising the clinical samples described in Example 1. The first algorithm involves counting of both urothelial and squamous cells with positive nuclear staining for telomerase. A positive test result is determined based on the percentage of cells stained. The second algorithm involves assessing morphological changes in urothelial cells in combination with positive nuclear staining for telomerase to derive a positive test result.

### Reading Algorithm—5% Cut Off

Slides were assessed at a magnification of ×200-400 and visually scanned across an adequate number of fields of view to identify more than 20 urothelial cells. The following cell numbers were recorded:
1. The number of urothelial cells showing nuclear staining.
2. The number of urothelial cells identified/evaluated in the process of recording point (1) above.

The following particulars were also noted:
1. Urothelial staining characteristics (nuclear/cytoplasmic).
2. Percentage of squamous cells showing nuclear staining (including both cells with or without cytoplasmic staining).
3. Total number of squamous epithelial cells evaluated.

TABLE 8
Cytological assessment of cell morphology and telomerase immunostaining
results in clinically validated samples (biopsy proven)

| | | Cytological | Anti-telomerase antibody test result | | | | | | |
|---|---|---|---|---|---|---|---|---|---|
| | | assessment | | | | Santa | Santa | Rockland | |
| Sienna | Clinical | of cell | Sienna | Novus | Novus NB | Cruz SC- | Cruz SC- | 600-401- | MBL |
| ID | status | morphology* | SCD-A7 | 2C4 | 100-297 | 377511 | 7212 | 252 | M216-3 |
| AUA14- | Negative | Atypical | Negative | Negative | Negative | Negative | Negative | Negative | Negative |
| 151 | | | | | | | | | |
| AUA14- | Positive | Atypical | Positive | # | # | # | # | # | Positive |
| 160 | | | | | | | | | |
| AUA14- | Positive | Atypical | Positive | Positive | Positive | Positive | Positive | Positive | Positive |
| 168 | | | | | | | | | |
| AUA14- | Positive | Atypical | Positive | # | # | Positive | Positive | # | # |
| 173 | | | | | | | | | |
| AUA14- | Positive | Atypical | Positive | Positive | Positive | Positive | Positive | Positive | Negative |
| 187 | | | | | | | | | |
| RMH13- | Positive | Atypical | Positive | # | Positive | # | # | # | # |
| 058 | | | | | | | | | |
| RMH13- | Positive | Atypical | Positive | Positive | # | # | # | Positive | # |
| 069 | | | | | | | | | |

| | | | | | | | |
|---|---|---|---|---|---|---|---|
| Correctly detected cancer % | 7/7 | 4/4 | 4/4 | 4/4 | 4/4 | 4/4 | 3/4 |

% - 2 samples excluded from analysis as clinical status could not be determined; AUA14-158; AUA14-172.
# - Insufficient material available to stain.
*An “atypical” cytological assessment of cell morphology indicates that cells in the sample have lost their normal appearance but have not reached the level of abnormality of malignant cells.

For this algorithm, a positive test result was defined as a slide in which more than 5% of the urothelial cells demonstrated positive nuclear staining (i.e. more than about 2 to 3 cells with positive nuclear staining per 20 urothelial cells).

### Reading Algorithm—Morphology Based

Slides were assessed to identify urothelial cells demonstrating morphological atypia (e.g. high nuclear to cytoplasmic ratio, nuclear chromatin variation, irregular nuclear outlines). Cells demonstrating morphological atypia were then assessed for the presence or absence of a positive immunocytochemistry signal.

Corresponding Papanicolau stained urine preparations were examined in conjunction with the telomerase immunocytochemistry slide during evaluation of clinical samples.

For this algorithm, a positive test result was defined as a slide in which the urothelial cells demonstrated morphological atypia in the presence of a positive nuclear stain.

### Comparison of Reading Algorithms

Comparison of the above reading algorithms is summarised in Table 9. Review of slides using morphology in combination with telomerase positive staining of cells resulted in an overall sensitivity of 83.3% whereas the 5% Cut off Algorithm resulted in an overall sensitivity of 57.1%. The sensitivity for the detection of Low Grade urothelial carcinoma increased to 75.0% using the morphology based reading algorithm in comparison to 50.0% achieved with the 5% cut off algorithm.

The morphology based algorithm demonstrates increased sensitivity and specificity for the overall detection of urothelial carcinoma as well as increased sensitivity and specificity for both high and low grade disease classifications in comparison to the 5% cut off algorithm.

Nonetheless, the 5% cut off algorithm also provided an effective approach for resolving an inconclusive cytological assessment in the analysed bladder cancer samples. For example, it is anticipated that 1 cell with positive nuclear staining per 20 urothelial cells indicates a positive test result.

TABLE 9
Comparison of the 5% Cut off and Morphology Based Algorithm results.

| | 5% Cut Off Algorithm | | | Morphology Based Algorithm | | |
|---|---|---|---|---|---|---|
| | | High Grade | Low Grade | | High Grade | Low Grade |
| Test Statistics | Overall | Disease | Disease | Overall | Disease | Disease |
| Number | 31 | 20 | 27 | 27 | 18 | 23 |
| Sensitivity | 57.1% | 100.0% | 50.0% | 83.3% | 100.0% | 75.0% |
| Specificity | 58.8% | 58.8% | 58.8% | 86.7% | 86.7% | 86.7% |
| Likelihood Ratio for | 1.39 | 2.43 | 1.21 | 6.25 | 7.50 | 5.63 |
| Positive Test (LR+) | | | | | | |
| Likelihood Ratio for | 0.73 | 0.00 | 0.85 | 0.19 | 0.00 | 0.29 |
| Negative Test (LR−) | | | | | | |
| Positive Predictive | 53.3% | 30.0% | 41.7% | 83.3% | 60.0% | 75.0% |
| Value (PPV) | | | | | | |
| Negative Predictive | 62.5% | 100.0% | 66.7% | 86.7% | 100.0% | 86.7% |
| Value (NPV) | | | | | | |
| Diagnostic Accuracy | 58.1% | 65.0% | 55.6% | 85.2% | 88.9% | 82.6% |

### Example 13: Identifying Morphologically Normal Malignant Cells a Histological Sample

The methods of the present application may be used to determine whether a subject has cancer where a histological assessment of cell morphology is negative for cancer.

Formalin fixed, paraffin embedded tissue (4 μM mounted on to glass slides) was obtained from an ethics-approved tissue biobank.

For the automated immunostaining process, slides were placed on the automated staining platform and the tissue was adhered to the slide by baking at 60° C. for 28 minutes. The deparaffinisation step was selected to remove the wax from the slide. Antigen retrieved was performed at 95° C. for 32 minutes. Post retrieval, the Pre-primary Peroxide Inhibitor was applied, prior to incubating with the telomerase-specific primary anti-hTERT antibody at 37° C. for 28 minutes. The Ventana Discovery reagent (Ventana Medical Systems, Inc, Cat No: 760-108) was required for a post-blocking step of 16 minutes. The remaining steps relied on standard Ventana platform settings using Ventana OptiView DAB IHC Detection Kit (Ventana Medical Systems Inc, Cat No: 760-700). Hematoxylin counterstaining was performed using Hematoxylin II (Ventana Medical Systems Inc, Cat No: 790-2208).

Samples from positive cancer patients (tumour and matched normal sample) were evaluated by looking for the presence or absence of telomerase in clinically relevant cells. The tumour samples were confirmed to contain malignant cells based on morphological assessment. The matched normal samples contained morphologically normal cells. A skin tumour sample (09RMH525 1F) showed positive telomerase staining in malignant squamous cells, while telomerase was also detected in morphologically normal squamous cells in the corresponding matched sample (09RMH525 2A). Both samples also contained non-clinically relevant cells that stained positive with the anti-hTERT antibody which could be distinguished from the clinically relevant cells based on morphology.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

The present application claims priority from AU 2015903361 filed 19 Aug. 2015, the disclosure of which are incorporated herein by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Ausubel et al. (1988) Current Protocols in Molecular Biology, Greene Pub. Associates and Wiley-Interscience (including all updates until present).
Blackburn et al. (2005) Molecular Cancer Research 3, 477-482.
Bodnar et al. (1998) Science 279, 349-52.
Brown (1991) Essential Molecular Biology: A Practical Approach, Volumes 1 and 2, IRL Press.
Coligan et al. (1994) Current Protocols in Immunology, John Wiley & Sons (including all updates until present). Glover et al. (1991) DNA Cloning: A Practical Approach, Volumes 1-4, IRL
Harlow and Lane (1988) Antibodies: A Laboratory Manual, Cold Spring Harbour Laboratory.
Leong and Zhuang, (2011) Pathobiology, 78(2), 99-114.
Perbal (1984) A Practical Guide to Molecular Cloning, John Wiley and Sons.
Masutomi et al., (2003) Cell, 114 (2), 241-253.
Sambrook et al. (1989) Molecular Cloning: A Laboratory Manual, Cold Spring Harbour Laboratory Press.

1. A method of identifying malignant cells in a sample obtained from a subject when a pathological assessment of cell morphology performed on the sample is negative for cancer, the method comprising, contacting cells from the sample with an anti-telomerase antibody and performing a pathological assessment of the cells to detect binding of the antibody to clinically relevant cells, wherein binding of the antibody to clinically relevant cells indicates the presence of malignant cells.

2. A method of determining whether a subject has cancer
(a) when a pathological assessment of cell morphology performed on a sample obtained from the subject is negative for cancer, the method comprising:
i) contacting a sample obtained from the subject with an anti-telomerase antibody;
ii) performing a pathological assessment of the sample to detect binding of the antibody to clinically relevant cells in the sample;
wherein binding of the antibody to one or more clinically relevant cells in the sample indicates that the subject has cancer, or
(b) the method comprising:
i) performing a pathological assessment of cell morphology on a sample obtained from the subject to determine the morphology of one or more clinically relevant cells in the sample;
ii) contacting a sample from the subject with an anti-telomerase antibody and performing a pathological assessment of the sample to detect binding of the antibody to clinically relevant cells in the sample;
wherein when the assessment of cell morphology is negative for cancer, binding of the antibody to one or more clinically relevant cells indicates that the subject has cancer.

3. (canceled)

4. The method of claim 1, wherein the pathological assessment of cell morphology is normal, pre-malignant, metaplastic or dysplastic.

5. (canceled)

6. The method of claim 1, wherein the clinically relevant cells comprise morphologically normal cells.

7. The method of claim 1, wherein the clinically relevant cells comprise pre-malignant, metaplastic and/or dysplastic cells.

8. The method of claim 3, wherein the pathological assessment to determine cell morphology and the pathological assessment to detect binding of the antibody to clinically relevant cells are performed simultaneously on the same cells.

9.-12. (canceled)

13. The method of claim 1, the method further comprising prescribing treatment of the subject for cancer when binding of the antibody to clinically relevant cells is detected.

14. The method of claim 1, wherein the anti-telomerase antibody is monoclonal, polyclonal, bispecific, chimeric, recombinant, anti-idiotypic, humanized, single-chain antibody molecule, or antigen-binding fragments thereof.

15. The method of claim 1, wherein the pathological assessment is a cytological assessment.

16. The method of claim 15, wherein the sample is a fluid sample.

17. (canceled)

18. The method of claim 15, wherein the cancer is selected from the group consisting of bladder cancer, thyroid cancer, breast cancer, cervical cancer.

19. (canceled)

20. The method of claim 1, wherein the pathological assessment is a histological assessment.

21. The method of claim 20, wherein the sample is a tissue sample.

22. The method of claim 21, wherein the tissue sample is selected from the group consisting of bladder, pancreas, liver, gall bladder, thyroid, ovary, lymph node, breast, cervix, lung, biliary tree, pancreas, lung, kidney, prostate, colon, stomach, oesophagus and brain.

23. The method of claim 21, wherein the cancer is selected from the group consisting of bladder cancer, pancreatic cancer, liver cancer, gall bladder cancer, thyroid cancer, breast cancer, lung cancer, mesothelioma, cervical cancer, ovarian cancer, kidney cancer, prostate cancer, colorectal cancer, stomach cancer, oesophageal cancer, brain cancer.

24.-26. (canceled)

27. A method comprising:
prescribing treatment of a subject for cancer,
wherein a pathological assessment of cell morphology performed on a sample from the subject is negative for cancer, and
wherein an anti-telomerase antibody binds to one or more clinically relevant cells in sample from the subject.
